(19) United States
(12) Patent Application Publication
(10) Pub. No.: US 2025/0375098 A1
(43) Pub. Date: Dec. 11, 2025

(54) SHEATHS FOR MEDICAL DEVICES AND RELATED METHODS

(71) Applicant: Boston Scientific Scimed, Inc., Maple Grove, MN (US)

(72) Inventors: Anthony Frank TASSONI, JR., Andover, MN (US); Gene Thomas STORBECK, Millis, MA (US); John D. KROEGER, Crosslake, MN (US); Brian T. BERG, St. Paul, MN (US)

(73) Assignee: Boston Scientific Scimed, Inc., Maple Grove, MN (US)

(21) Appl. No.: 19/233,209

(22) Filed: Jun. 10, 2025

Related U.S. Application Data
(60) Provisional application No. 63/658,671, filed on Jun. 11, 2024.

(51) Int. Cl. A61B 1/00; A61B 1/005; A61M 25/00

(52) U.S. Cl. CPC A61B 1/0011; A61B 1/005; A61M 25/0009; A61M 25/0045; A61M 25/005

(57) ABSTRACT

Sheaths for medical devices and related methods are described. A method of manufacturing a medical device may include providing a sheath. The method may further include providing a tube. The tube may include an inner first layer and an outer second layer. The first layer may be more susceptible to melting than the second layer. The method may include positioning the tube around the sheath such that the first layer is closer to the sheath than the second layer is. The method may further include applying heat to the tube or the sheath such that the first layer penetrates the sheath.

### CROSS-REFERENCE TO RELATED APPLICATION(S)

This application claims the benefit of priority to U.S. Provisional Application No. 63/658,671, filed on Jun. 11, 2024, which is incorporated by reference herein in its entirety.

### TECHNICAL FIELD

Aspects of the present disclosure generally relate to medical devices and procedures. In particular, aspect of the present disclosure relate to sheaths for medical devices.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Endoscopic procedures may be carried out by inserting an insertion device into a patient's body through a surgical incision, or via a natural orifice (e.g., mouth, vagina, or rectum).

The insertion device includes a shaft that includes one or more lumens or working channels therethough. A lumen (or lumens) may often receive various devices and structures such as medical instruments (e.g. irrigation tubes, aspiration tubes, forceps, electrosurgical knives, brushes, RF electrodes, and/or other tools) designed to be operated at a distal end of an insertion device. The insertion device also includes a sheath surrounding the shaft. The sheath may include multiple layers, such as braids and coatings.

### SUMMARY

Each of the aspects disclosed herein may include one or more of the features described in connection with any of the other disclosed aspects.

Aspects of the disclosure may related to sheaths for medical devices and related methods. For example, the disclosure includes a method for manufacturing a medical device. The method may include providing a sheath, providing a tube, positioning the tube around the sheath, and applying heat to the tube or sheath such that the first layer penetrates into the sheath. The tube may include an inner first layer and an outer second layer. The first layer may be more susceptible to melting than the second layer. The tube may be positioned so that the first layer is closer to the sheath than the second layer is.

The method may include one or more of the following features. For example, the sheath may be a braided sheath. Applying heat to the tube or the sheath may cause the first layer to penetrate into open spaces of the braided sheath. The first layer may include a non-cross linked polymer and the second layer may include a cross linked polymer. The tube make include an adhesives layer between the first layer and the second layer. The first layer may be chemically bonded to the second layer. As heat is applied to the tube, the second layer may contact the sheath and inhibit the first layer from flowing deeper into the sheath. The sheath may have a sheath thickness and the first layer may have a first layer thickness. The first layer thickness may be equal to or less than the sheath thickness. The first layer may include a first material and the second layer may include a second material. Each of the first material and the second material may be in the same family of elastomers. The tube may be positioned directly on the sheath such that the tube directly contacts the sheath. The second layer may include a greater molecular weight than the first layer. The second layer may have a higher durometer than the first layer. The first layer may include an inhibitor. The second layer may be vulcanized. Heat may be applied to the sheath such that the sheath inductively heats the tube.

According to some aspects, the disclosure may include a medical device shaft. For example, the medical device shaft may include a braided sheath. The medical device shaft may further include an inner first layer and an outer second layer. The first layer may be more susceptible to melting than the second layer. The first layer may be bonded to the second layer by an adhesive or a chemical bond. The first layer may extend at least partially through a thickness of the braided sheath. An inner surface of the second layer may contact an outer surface of the braided sheath.

The medical device shaft may include one or more of the following features. For example, a material of the first layer may be more susceptible to melting than a material of the second layer. The second layer may be formed from a material configured to shrink when exposed to a predetermined temperature. The first layer may be formed from a material configured to become flowable when exposed to the predetermined temperature.

According to some aspects, the disclosure may include another medical device shaft. For example, the medical device shaft may include a coil or flexible tube, a braided sheath radially surrounding the coil or flexible tube, an inner first layer compressing a non-crosslinked polymer and an outer second layer comprising a crosslinked polymer. The first layer may be bonded to the second layer. The first layer may extend at least partially through a thickness of the braided sheath and terminates at a point radially outward of an outer surface of the coil or flexible tube. An inner surface of the second layer may contact an outer surface of the braided sheath.

The medical device shaft may include one or more of the following features. For example, the first layer may include an inhibitor or the second layer may be vulcanized.

### BRIEF DESCRIPTION OF THE FIGURES

The accompanying drawings, which are incorporated in and constitute a part of this specification, illustrate examples of this disclosure and together with the description, serve to explain the principles of the disclosure.

FIG. 1 depicts an exemplary medical device.

FIG. 2 depicts a longitudinal cross-sectional view of a portion of an exemplary medical device.

FIG. 3 depicts a cross-sectional view of a portion of the medical device of FIG. 1.

FIG. 4A depicts a lateral cross-sectional view of a portion of the medical device of FIG. 1 in a first configuration.

FIG. 4B depicts a lateral cross-sectional view of a portion of the medical device of FIG. 1 in a second configuration.

FIG. 4C depicts a lateral cross-sectional view of a portion of the medical device of FIG. 1 in a third configuration.

FIG. 5 depicts an exemplary method flowchart.

### DETAILED DESCRIPTION OF THE FIGURES

Reference will now be made in detail to examples of the disclosure described above

and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device. In contrast, “distal” refers to a position relatively further away from the medical professional using the medical device, or closer to the interior of the body. The term “diameter” encompasses widths for non-circular elements. As used herein, the terms “comprises,” “comprising,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

A shaft of a medical device may include a sheath, which may include a plurality of layers. For example, the sheath may include a coil, a braided (or otherwise woven) tube (which may be referred to as a braid) and one or more layers of coatings outside of the braid. Although coils and braids are referred to herein, it will be appreciated that alternative sheaths may also be utilized (e.g, extruded sheaths, molded sheaths, etc.). The materials of the coatings may be positioned over the braided tube. Conventional sheaths may be formed by extruding coatings and/or by using heat-shrink layers of material. When the coatings materials are manufactured onto the braided tube in conventional sheaths, complications may arise when the materials flow into/penetrate in irregular patterns into the braided tube. Penetration of extruded materials may vary based on inputs that need adjustment during a manufacturing process. Heat-shrinkable sheets of material may have variable properties from batch to batch or within batches. The variability of the deposition of conventional coatings may cause the coatings to penetrate entirely through the braid, contacting the coil or other elements that are radially inside of the braid. Different portions of a given conventional sheath may have different stiffnesses as a result of varying levels of penetration of the coatings. Furthermore, a stiffness may vary from shaft to shaft or along a length of an individual shaft using conventional manufacturing methods, creating undesired variability between devices.

The shafts disclosed herein include a braided tube and at least two layers of coatings on an outer surface of the braided tube. Prior to deposition on the braid, the plurality coatings may be in a single sheet of material (e.g., a single tube of material). A first material may be on a radially inner portion of the tube, and a second layer of material may be on a radially outer portion of the tube. The first material may be a higher melt (more easily melting) material than the second material. In other words, the second material may be a lower melt (less easily melting) material than the first material. Heat may be applied to the tube, so that the first material melts into the braid. The first material may be flowable into the braid. The second material may not flow into the braid. The second material may precisely control how much material flows into the braid, because the flow may cease when the second material contacts a radially outer surface of the braid.

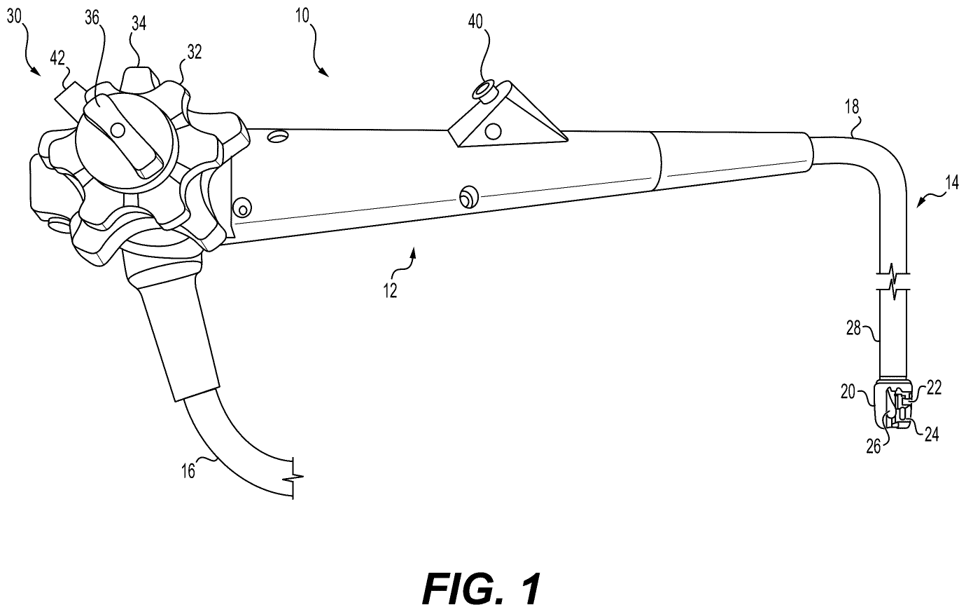

FIG. 1 depicts an exemplary medical device 10 having a handle 12 and an insertion portion 14. Medical device 10 may also include an umbilicus 16 for purposes of connecting medical device 10 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 18 and a distal tip 20. Distal tip 20 may include an imaging device 22 (e.g., a camera) and a lighting source 24 (e.g., an LED or an optical fiber). Distal tip 20 may be side-facing. That is, imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device 22 may face distally (approximately parallel to a longitudinal axis of distal tip 20/shaft 18).

Distal tip 20 may also include an elevator 26 for changing an orientation of an accessory device or a tool inserted in a working channel of medical device 10. Elevator 26 may alternatively be referred to as a swing stand, pivot stand, raising base, or any suitable other term. Elevator 26 may be pivotable via, e.g., an actuation wire or another control element that extends from handle 12, through shaft 18, to elevator 26.

A distal portion of shaft 18 that is connected to distal tip 20 may have a steerable section 28. Steerable section 28 may be, for example, an articulation joint. Shaft 18 and steerable section 28 may include a variety of structures which are known or may become known in the art.

Handle 12 may have one or more actuators/control mechanisms 30. One or more of control mechanisms 30 may provide control over steerable section 28. One or more of control mechanisms may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36, 42 (e.g., knobs or levers) for preventing steering and/or braking of steerable section 28 in at least one of an up, down, left, or right direction. Handle 12 may include an elevator control lever (not shown). The elevator control lever may raise and/or lower elevator 26, via connection between the lever and an actuating wire that extends from the lever, through shaft 18, to elevator 26. A port 40 may allow passage of a tool through port 40, into a working channel of the medical device 10, through shaft 18, to distal tip 20.

In use, an operator may insert at least a portion of shaft 18 into a body lumen of a subject. Distal tip 20 may be navigated to a procedure site in the body lumen. The operator may insert an accessory device (not shown) into port 40, and pass the accessory device through shaft 18 via a working channel to distal tip 20. The accessory device may exit the working channel at distal tip 20. The user may use elevator control lever to raise elevator 26 and angle the accessory device toward a desired location (e.g., a papilla of the pancreatico-biliary tract). The user may use the accessory device to perform a medical procedure.

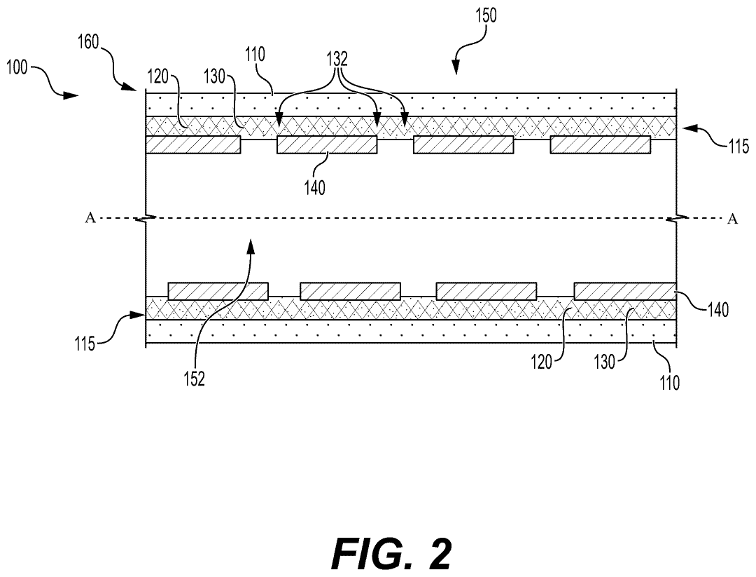

FIG. 2 illustrates aspects of an exemplary medical device 100, having any of the properties of medical device 10. Specifically, FIG. 2 depicts a longitudinal cross section (a cross-section taken along a central longitudinal axis A) of a portion of medical device 100. Medical device 100 may include handle 12 (not shown in FIG. 2) and a shaft 150. Shaft 150 may have any of the properties of shaft 18, discussed above. Shaft 150 may be generally tubular, and may extend from a distal end of handle 12. Shaft 150 may include a proximal end that is positioned at the distal end of handle 12 and a distal end. In FIG. 1, the longitudinal cross-section of medical device 100 depicts shaft 150 as being having a hollow central lumen 152; however it should be understood that an interior shaft 150 may be solid, or hollow, and/or may define one or more working channels, fluid channels and lumens that extend through, e.g., lumen 152. For example, one of the one or more fluid channels may be a fluid delivery channel, and another of the one or more fluid channels may be a working channel and/or suction or negative pressure channel.

Still referring to FIG. 2, medical device 100 may include a coil 140. Coil 140 may radially surround and be approximately coaxial with lumen 152. Coil 140 may extend from a proximal portion of shaft 150 to a distal portion of shaft 150 or along only a portion of a length of shaft 150. Coil 140 may include a variable pitch over a longitudinal length of coil 140. Coil 140 may have properties of any coils of shafts known in the art. For example, coil 140 may be a flat coil comprised of a plurality of windings. In some examples, coil 140 may be omitted or replaced with a flexible tube.

Shaft 150 may further include a braid 130, which may be a sheath. In alternatives, other types of liners or sheaths may be used in lieu of braid 130. Braid 130 may radially surround and be coaxial with lumen 152 and coil 140. An inner diameter (inner surface) of braid 130 may be flush with an outer diameter (outer surface) of coil 140. Braid 130 may be a braided tube/sheath including a braiding or another type of weaving. The braiding of braid 130 may include a plurality of gaps or open spaces 132. Braid 130 may include a thickness between an inner diameter (inner surface) of braid 130 and an outer diameter (outer surface) of braid 130. Braid 130 may be configured to be heated (e.g., may be formed of a metallic material that will transmit heat to other portions of shaft 150, as discussed below.)

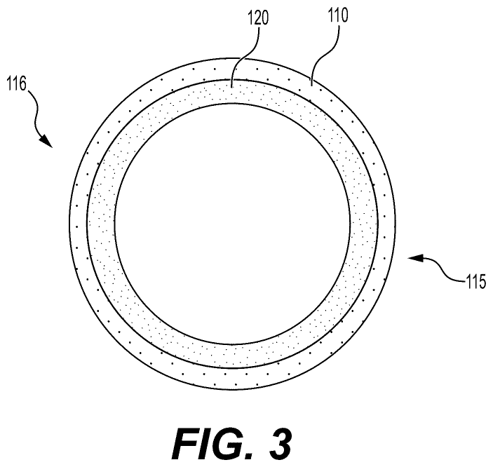

As shown in FIG. 2, medical device 100 may further include a multi-layered coating 115. Coating 115 may radially surround and be approximately coaxial with lumen 152. Together, coil 140, braid 130, and coating 115 may form a sheath 160. Coating 115 may include a first, inner layer 120 and a second, outer layer 110. First layer 120 and second layer 110 may each include an inner diameter (defined by an inner surface or boundary) and an outer diameter (defined by an outer surface or boundary). As shown in FIG. 3, prior to manufacturing shaft 150, coating 115 may be a tube 116 or other sheet of material including both first layer 120 and second layer 110. FIG. 3 shows a cross-section of tube 116. Although coatings 115 with only two layers 110, 120 are described herein, it will be appreciated that coatings 115 may include three or more layers.

To form tube 116, an inner diameter (inner surface) of second layer 110 may be chemically bonded, bonded, adhered to, extruded onto, reflowed onto, integrated with or otherwise fixed to an outer diameter (outer surface) of first layer 120. Second layer 110 may remain bonded or otherwise fixed to first layer 120 after tube 116 has been heated to a predetermined temperature. The predetermined temperature is further discussed below. Although one or more components (e.g., second layer 110, first layer 120, braid 130, coil 140, and coating 115) of medical device 100 are described in this disclosure as including a diameter, it should be understood that the components of medical device 100 may include any shape and any dimension and that the term “diameter” encompasses widths of non-circular components. According to some aspects, tube 116 may include an adhesive layer (not shown) positioned between first layer 120 and second layer 110 and configured to adhere first layer 120 to second layer 110. In some examples, tube 116 may be formed by extruding one of first layer 120 or second layer 110 on the other of first layer 120 or second layer 110. In alternatives, first layer 120 and second layer 110 may be separately formed, and first layer 120 and second layer 110 may be fixed to one another (e.g., via adhesive). In some examples, first layer 120 may have an inhibitor or second layer 110 may have vulcanization to keep first layer 120 and second layer 110 separate from one another.

First layer 120 may be more susceptible to melting than second layer 110. In other words, first layer 120 may be more high melt, while second layer 110 may be more low melt. For example, second layer 110 may have a higher melting point than first layer 120, such that when first layer 120 and second layer 110 are heated to the predetermined temperature (e.g., the melting point of first layer 120), second layer 110 remains solid and/or non-flowable. In other examples, the predetermined temperature may be one at which second layer melts but does not flow into braid 130, as discussed below.

In some examples, second layer 110 may comprise a cross linked polymer, such as, but not limited to, cross-linked polyether block amide (e.g., PEBAX®), nylon, arnitel, thermoplastic elastomers (e.g., Hytrel®), other polymers, and other materials with similar properties or materials used by those skilled in the art. According to some aspects of the disclosure, second layer 110 may comprise a non-cross linked polymer such as fluorinated ethylene propylene (FEP), polytetrafluoroethylene (PTFE), polyether ether ketone (PEEK), or other similar materials, which may have heat shrink properties. First layer 120 may comprise a non-cross linked polymer, such as PEBAX or polyurethane. According to some aspects of the disclosure, second layer 110 may include a greater durometer value than first layer 120. In aspects, a durometer of second layer 110 may be only slightly greater than a durometer of first layer 120. In some examples, a material of second layer 110 may have a higher molecular weight than a material of first layer 120. According to one or more aspects of the disclosure, second layer 110 and first layer 120 may be formed from a material from the same family of elastomers. For example, both first layer 120 and second layer 110 may be formed of, for example, PEBAX, with second layer 110 having a different (e.g., greater) durometer than first layer 120. First layer 120 and second layer 110 may each be formed from resin. According to one or more aspects of the disclosure, the material of second layer 110 may include a polymer composition that imparts a higher stiffness (when compared to first layer 120 or other materials of second layer 110). In some examples, the polymer composition of second layer 110 may cause second layer 110 to have a higher viscosity, softening temperature, and/or melting point.

As discussed above, tube 116 may include more than two layers of materials. Additional layers of material may be radially over or under layers 110, 120. Alternatively, different materials may be used at different longitudinal locations and/or circumferential locations along tube 116. For example, layers 110, 120 may have different material properties along a longitudinal length of tube 116. Such different properties may provide different stiffnesses of sheath 160 along a longitudinal length of sheath 160. In other examples, layers 110, 120 may have varying properties circumferentially around tube 116. Such different properties may provide different stiffnesses of sheath 160 around a circumference of shaft 150 and may, for example, bias sheath 160 to a particular bent shape. In some examples, variations in layers 110, 120 may be variations in thicknesses of layers 110, 120 longitudinally and/or circumferentially. For example, one or more of the thicknesses of layers 110, 120 may decrease in thickness in the proximal to distal direction along axis A. In some examples, the thicknesses of layers 110, 120 may both decrease in thickness in the proximal to distal direction along axis A. In other examples, different materials may be used at different longitudinal and/or circumferential locations along tube 116.

During manufacture of shaft 150, tube 116 of coating 115 may be positioned over and/or radially surrounding braid 130. Heating coating 115 to a predetermined temperature may cause first layer 120 to flow through gaps/open spaces 132 of the braiding of braid 130 (e.g. flow through braid 130 radially inward toward axis A). First layer 120 may thus reinforce braid 130. For example, the predetermined temperature may be the melting point of first layer 120 or a temperature above the melting point of first layer 120. According to some aspect, the predetermined temperature may be greater than the melting point of first layer 120 but lesser than a melting point of second layer 110. In other examples, the predetermined temperature may be greater than the melting point of second layer 110 but second layer 110 may still not be flowable enough to flow into open spaces 132. While being heated, coating 115 may decrease in diameter until the inner diameter (inner surface) of second layer 110 contacts and/or is flush with the outer diameter (outer surface) of braid 130, with first layer 120 having flowed into open spaces 132 of braid 130. For example, second layer 110 may be formed of a crosslinked material that shrinks when heat is applied. First layer 120 may comprise a non-crosslinked polymer, such as polyether block amide (PEBAX), polyurethanes, and other similar polymers. When heated to the predetermined temperature, first layer 120 may become flowable through the open spaces 132 braiding of braid 130 (e.g., the thickness of braid 130).

Prior to manufacturing shaft 150, first layer 120 in tube 116 may include a thickness between its inner diameter (radially inner surface or boundary) and its outer diameter (radially outer surface or boundary). The thickness of first layer 120 may be sized, or otherwise configured, so that, during manufacture of shaft 150, first layer 120 flows through the thickness of braid 130 and terminates at the inner diameter (inner surface) of braid 130 and/or outward of the outer diameter (outer surface) of coil 140. In other words, the thickness of first layer 120 may be sized, or otherwise configured, so that the thickness of first layer 120 includes a sufficient amount of material so that first layer 120 may flow through braid 130 up until the inner diameter (inner surface) of braid 130. In other examples, first layer 120 may have an innermost edge that is radially outward of an inner diameter (inner surface) of braid 130. In other words, first layer 120 may not fully penetrate through braid 130. A thickness of first layer 120 may be chosen to provide a desired (e.g., predetermined) flexibility of shaft 150. According to some embodiments, the thickness of first layer 120 may be less than or equal to the thickness of braid 130 so as to inhibit (e.g., prevent) first layer 120 from flowing beyond the inner diameter (inner surface) of braid 130.

A thickness of first layer 120 may be chosen to correspond to a desired stiffness of shaft 150. For example, for at least some shafts 150, it may be undesirable for first layer 120 to flow beyond the inner diameter (inner surface) of braid 130 as first layer 120 may contact and/or adhere to coil 140, or other internal components of medical device, and cause braid 130 and medical device 100 to become too stiff. Conversely, for such shafts, it may also be undesirable for first layer 120 to not flow a sufficient distance through the thickness of braid 130, resulting in a braid 130 and medical device 100 that is too flexible (e.g., not stiff enough).

Accordingly, the thickness of first layer 120 relative to the thickness of braid 130 may control the stiffness and flexibility of braid 130 after first layer 120 has flowed through a portion (e.g., at least partially) or an entirety of the thickness of braid 130 and has been allowed to cool and/or cure. Various thicknesses and/or materials of first layer 120 and second layer 110 may be chosen in order to produce shaft 150 with a desired stiffness. Using tube 116 (or a film including a first layer and a second layer as described below) may help to produce consistent stiffness within a particular shaft 150 and across different shafts 150. As compared with extrusion of an outer cover (where parameters may be required to be adjusted to maintain consistent shaft stiffness) or traditional heat-shrink manufacturing (where the heat shrink material may have inconsistent properties), using tube 116 may provide for a consistent penetration of first layer 120 within braid 130 (e.g., a consistent depth in which first layer 120 penetrates through braid 130 toward a central longitudinal axis of medical device 100), a consistent thickness of second layer 110 outside of braid 130, and, thus, a consistent stiffness of shaft 150.

Second layer 110 may be configured to shrink/decrease when exposed to heat, such as the predetermined temperature. For example, when heated to the predetermined temperature, second layer 110 may begin to shrink. As second layer 110 is shrinking, second layer 110 may push first layer 120 into braid 130 and first layer 120 may flow (e.g., extend through) through braid 130 until the inner diameter (inner surface) of second layer 110 contacts the outer diameter (outer surface) of braid 130. A bonding or adhesion between second layer 110 and first layer 120 may cause first layer 120 to stop further flow through braid 130 when second layer 110 contacts braid 130. For example, after the inner diameter (inner surface) of second layer 110 contacts braid 130, second layer 110 may inhibit first layer 120 from flowing deeper into braid 130, relative to axis A, and halt first layer 120 in place. In another example, if second layer 110 contacts braid 130 while first layer 120 is at a midpoint of the thickness of braid 130, first layer 120 will remain at the midpoint and discontinue flowing toward the inner diameter (inner surface) of braid 130 because the force applied by the shrinkage of second layer 110 stops and/or hoop stress of second layer 110 stops.

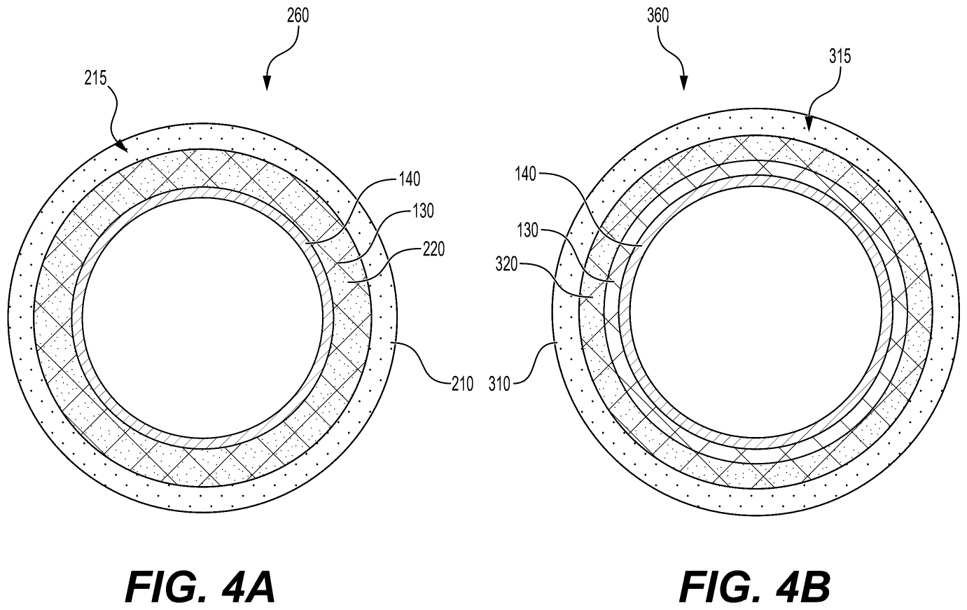

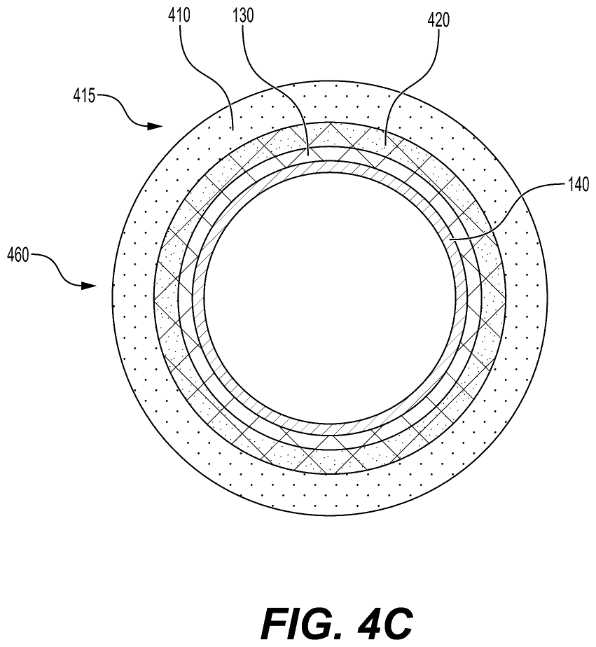

FIGS. 4A-4C show exemplary sheaths having different properties (e.g., stiffness) resulting from having different coatings. Materials and dimensions of coatings may be chosen in order to produce a shaft with desired properties. The examples provided below are merely illustrative, and coatings with other properties may be chosen to impart different properties. Furthermore, although each of the sheaths below include braid 130 and coil 140, one or both of braid 130 and coil 140 may be omitted or substituted with an alternative element (e.g., coil 140 may be replaced with an alternative liner, such as a flexible tube).

FIG. 4A shows a first exemplary sheath 260. Sheath 260 may include braid 130, coil 140, and a coating 215, having any of the properties of coating 115. Prior to manufacture of sheath 260, coating 215 may be in the form of a tube, similar to tube 116, discussed above. Coating 215 may have an inner, first layer 220 (having any of the properties of first layer 120) and an outer, second layer 210 (having any of the properties of second layer 110). Braid 130 may have a thickness of approximately 0.010-0.030 inches, or approximately 0.02 inches, although such a dimension is merely exemplary. Once first layer 220 has flowed into open spaces 132 of braid 130, first layer 220 may have a thickness of approximately 0.010-0.030 inches, or approximately 0.02 inches, and/or the thickness of first layer 220 may include a sufficient amount of material to extend through an entirety of the thickness of braid 130. In other words, first layer 220 may extend approximately through an entire thickness of braid 130. An inner edge of first layer 220 may be adjacent to (e.g., abut) coil 140. Second layer 210 have a thickness of approximately 0.005 inches to approximately 0.025 inches, or approximately 0.010 inches to approximately 0.020 inches.

FIG. 4B shows a second exemplary sheath 360. Sheath 360 may include braid 130, coil 140, and a coating 315, having any of the properties of coating 115. Prior to manufacture of sheath 360, coating 315 may be in the form of a tube, similar to tube 116, discussed above. Coating 315 may have an inner, first layer 320 (having any of the properties of first layer 120) and an outer, second layer 310 (having any of the properties of second layer 110). Braid 130 may have a thickness of approximately 0.010-0.030 inches, or approximately 0.02 inches or any suitable alternative thickness. Once first layer 320 has flowed into open spaces 132 of braid 130, first layer 320 may have a thickness of approximately 0.005 inches to approximately 0.015 inches, or approximately 0.01 inches, and/or the thickness of first layer 320 may include an insufficient amount of material for first layer 320 to extend through an entirety of the thickness of braid 130. In other words, first layer 320 may extend approximately to the midpoint (or just pass the midpoint) of the thickness of braid 130 (e.g., first layer 320 may extend through only a portion of the thickness of braid 130). Second layer 310 have a thickness of approximately 0.005 inches to approximately 0.025 inches, or approximately 0.01 inches.

As shown in FIG. 4A, first layer 220 extends approximately through an entirety of the thickness of braiding 130. Conversely, as shown in FIG. 4B, first layer 320 extends through a portion of the thickness of braiding 130. It should be understood that sheath 260 would be more stiff than sheath 360 because first layer 220 extends through a greater portion of braiding 130 than first layer 320.

FIG. 4C shows a third exemplary sheath 460. Sheath 460 may include braid 130, coil 140, and a coating 415, having any of the properties of coating 115. Prior to manufacture of sheath 460, coating 415 may be in the form of a tube, similar to tube 116, discussed above. Coating 415 may have an inner, first layer 420 (having any of the properties of first layer 120) and an outer, second layer 410 (having any of the properties of second layer 110). Braid 130 may have a thickness of approximately 0.010-0.030 inches, or approximately 0.02 inches or any suitable alternative thickness. Once first layer 420 has flowed into open spaces 132 of braid 130, first layer 420 may have a thickness of approximately 0.005 inches to approximately 0.015 inches, or approximately 0.01 inches, and/or the thickness of first layer 420 may include an insufficient amount of material for first layer 420 to extend through an entirety of the thickness of braid 130. In other words, first layer 420 may extend approximately to the midpoint (or just past the midpoint) of the thickness of braid 130 (e.g., first layer 420 may extend through only a portion of the thickness of braid 130). Second layer 410 have a thickness of approximately 0.015 inches to approximately 0.040 inches, approximately 0.02 inches to approximately 0.035 inches, or any suitable thickness.

As compared with sheath 360, an increased thickness of second layer 410 versus second layer 310 may cause sheath 460 to have a greater stiffness. The aspects discussed above for FIGS. 4A-4C are merely exemplary to illustrate how thicknesses of layers of coatings 115, 215, 315, 415 may be varied to modulate stiffness of the sheaths disclosed herein.

According to one or more aspects of the disclosure, to prevent first layer 120 and second layer 110 from mixing or flowing into one another, when exposed to the predetermined temperature and/or when coating 115 is manufactured, first layer 120 may include an inhibitor. Similarly, according to one or more aspects of the disclosure, second layer 110 may be vulcanized to prevent first layer 120 and second layer 110 from mixing or flowing into one another when exposed to the predetermined temperature and/or when coating 115 is manufactured. Physical interactions (e.g., van der Waals forces, hydrogen bonding, dipole-dipole interactions, complementary properties such as nonpolar and polar, etc.) between first layer 120 and second layer 110, coextrusion of first layer 120 and second layer 110 and diffusion occurring during coextrusion, and chemical bonding may prevent first layer 120 and second layer 110 from mixing or flowing into one another when heated or manufactured. As described above, second layer 110 may be adhered to, glued to, reflowed onto the outer diameter (outer surface) of first layer 120, one or more of the listed methods of fixing second layer 110 to first layer 120 may prevent first layer 120 and second layer 110 from mixing or flowing into one another when heated or manufactured.

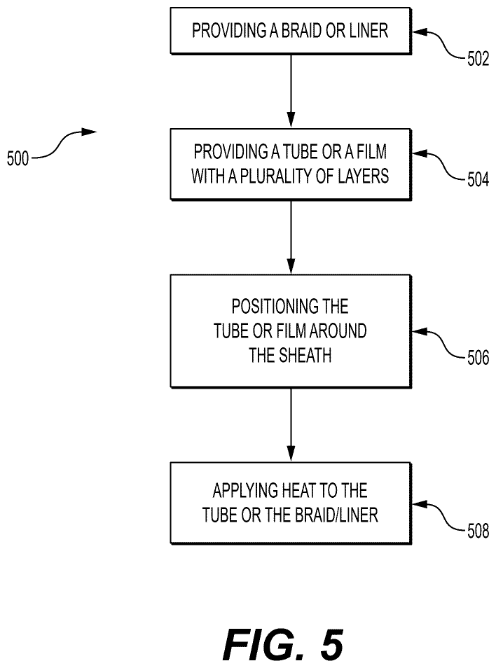

As seen in FIG. 5, this disclosure includes a method 500 of manufacturing a sheath (e.g., sheath 160, 260, or 360) medical device 100. Method 500 may include an initial step of providing a mandrel and a coil (e.g., coil 140) or other liner. Further, method 500 may include the initial step of positioning coil 140 (or another liner) along the mandrel such that coil 140 (or another liner) radially surrounds the mandrel. Coil 140 (or another liner) may be positioned so that it is coaxial with the mandrel. The inner diameter (inner surface) of coil 140 (or other liner) may contact and/or be flush with the mandrel.

Method 500 may include step 502 of providing braid 130 or another type of liner. Step 502 may further include positioning braid 130 or the other type of liner along coil 140 such that braid 130 radially surrounds coil 140 and the (optional) mandrel. An inner diameter (inner surface) of braid 130 may contact an outer diameter (outer surface) of coil 140.

Method 500 may include step 504 of providing a tube (e.g., tube 116), or a film, with a plurality of layers of material (e.g., first layer 120, 220, 320, or 420 and second layer 110, 210, 310, or 410).Although tube 116 of coating 115 is referenced herein, it will be appreciated that tubes of the alternative coatings (e.g., coating 215, 315, or 415) discussed above may be used. The film may be a sheet of the plurality of layers of material including the first layer and the second layer (e.g., first layer 120, 220, 320, or 420 and second layer 110, 210, 310, or 410). The film may be generally planar and may be flexible so that the film may be wrapped around braid 130 (e.g., an outer surface of braid 130). For example, a film may be used in a reel-to-reel type of manufacturing process, where multiple shafts are manufactured during a same manufacturing step.

Method 500 may further include step 506 of positioning tube 116 or the film around braid 130 or the alternative liner. Tube 116 or the film may be positioned along braid 130, radially surrounding braid 130 and coil 140. Tube 116 may be positioned such that first layer 120 of coating 115 is closer to braid 130 than second layer 110. Although first layer 120 and second layer 110 of coating 115 are referenced herein, it will be appreciated that the first layer and second layer of the alternative coatings (e.g., first layer 220, 320, 420 and second layer 210, 310, 410 respectively) discussed above may be used. As described above, the film may be wrapped around braid 130.

Method 500 may include step 508 of applying heat to the tube 116 (or the film) and/or braid 130 (or other liner). After tube 116 has been positioned along braid 130, heat may be applied to coating 115 at the predetermined temperature. For example, heat may be applied externally to coating 115 by another machine (such as, but not limited to, an oven) and/or coating 115 may be inductively heated by heating braid 130 or another liner. As described above, as coating 115 is heated, first layer 120 may become flowable. Second layer 110 may not flow or may not be flowable enough so as to pass through braid 130 or another liner (e.g., may not flow through open spaces 132).

In some examples, second layer 110 may shrink as heat is applied. A force generated from the shrinking of second layer 110 may be sufficient to push first layer 120 through the braiding of braid 130 or gaps of another liner. Second layer 110 may stop shrinking upon an inner diameter (inner surface of second layer 110) contacting the outer diameter (outer surface) of braid 130, or an operator may discontinue the application of heat to coating 115 to cease shrinking of second layer 110 and/or flowing of first layer 120. After second layer 110 contacts the outer diameter (outer surface) of braid 130, first layer 120 may discontinue flowing through the thickness of braid 130 and halt in place. In other words, second layer 110 contacting the outer diameter (outer surface) of braid 130 may control how far first layer 120 flows through the thickness of braid 130. After discontinuing heating, coating 115 may be allowed to cool and/or cure. In aspects where second layer 110 comprises a non-cross linked polymer, a compressive force (e.g., such as force generated by heat shrinkage) may be used to cause first layer 120 to flow through braid 130. In alternatives, an additional heat-shrink outer layer of material may be applied over second layer 110 to cause coating 115 to flow into braid 130.

While principles of this disclosure are described herein with reference to

illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A method of manufacturing a medical device, comprising:
providing a sheath;
providing a tube including an inner first layer and an outer second layer, wherein the inner first layer is more susceptible to melting than the outer second layer;
positioning the tube around the sheath, such that the inner first layer is closer to the sheath than the outer second layer is;
applying heat to the tube or the sheath, such that the inner first layer penetrates into the sheath.

2. The method of claim 1, wherein the sheath is a braided sheath, and wherein applying heat to the tube or the sheath causes the inner first layer to penetrate into open spaces of the braided sheath.

3. The method of claim 1, wherein the inner first layer includes a non-cross linked polymer.

4. The method of claim 3, wherein the outer second layer includes a cross linked polymer.

5. The method of claim 1, wherein the tube includes an adhesive layer between the inner first layer and the outer second layer.

6. The method of claim 1, wherein the inner first layer is chemically bonded to the outer second layer.

7. The method of claim 1, wherein, as heat is applied to the tube, the outer second layer contacts the sheath and inhibits the inner first layer from flowing deeper into the sheath.

8. The method device of claim 1, wherein the sheath has a sheath thickness and the inner first layer has an inner first layer thickness, wherein the inner first layer thickness is equal to or less than the sheath thickness.

9. The method of claim 1, wherein the inner first layer includes a first material, wherein the outer second layer includes a second material, and wherein each of the first material and the second material is from a same family of elastomers.

10. The method of claim 1, wherein the tube is positioned directly around the sheath, such that the tube directly contacts the sheath.

11. The method of claim 1, wherein the outer second layer includes a greater molecular weight than the inner first layer.

12. The method of claim 1, wherein the outer second layer has a higher durometer than the inner first layer.

13. The method of claim 1, wherein the inner first layer includes an inhibitor.

14. The method of claim 1, wherein the outer second layer is vulcanized.

15. The method of claim 1, wherein heat is applied to the sheath, such that the sheath inductively heats the tube.

16. A medical device shaft comprising:
a braided sheath;
a tube having an inner first layer and an outer second layer, wherein the inner first layer is more susceptible to melting than the outer second layer, and wherein the inner first layer is bonded to the outer second layer by an adhesive or a chemical bond;
wherein the inner first layer extends at least partially through a thickness of the braided sheath, wherein an inner surface of the outer second layer contacts an outer surface of the braided sheath.

17. The medical device of claim 16, wherein a material the inner first layer is more susceptible to melting than a material of the outer second layer.

18. The medical device of claim 16, wherein, the outer second layer is formed from a material configured to shrink when exposed to a predetermined temperature, and the inner first layer is formed from a material configured to become flowable when exposed to the predetermined temperature.

19. A medical device shaft comprising:
a coil or flexible tube;
a braided sheath radially surrounding the coil or flexible tube;
an inner first layer comprising a non-crosslinked polymer; and
an outer second layer comprising a crosslinked polymer, wherein the inner first layer is bonded to the outer second layer;
wherein the inner first layer extends at least partially through a thickness of the braided sheath and terminates at a point radially outward of an outer surface of the coil or flexible tube;
wherein an inner surface of the outer second layer contacts an outer surface of the braided sheath.

20. The medical device shaft of claim 19, wherein the inner first layer includes an inhibitor or the outer second layer is vulcanized.
